(19) United States
(12) Patent Application Publication
(10) Pub. No.: US 2026/0232320 A1
(43) Pub. Date: Aug. 13, 2026

(54) NANOFIBER NERVE CAPS TO INHIBIT NEUROMA FORMATION

(71) Applicant: The Johns Hopkins University, Baltimore, MD (US)

(72) Inventors: Hai-Quan MAO, Baltimore, MD (US); Sami TUFFAHA, Baltimore, MD (US); Ahmet HOKE, Towson, MD (US); Anson Y. ZHOU, Medford, NY (US); Bruce Peter ENZMANN, Virginia Beach, VA (US); Michael James LAN, Hockessin, DE (US); Erica LEE, Baltimore, MD (US); Ruifa MI, Baltimore, MD (US); Russell MARTIN, Baltimore, MD (US)

(21) Appl. No.: 19/155,754

(22) PCT Filed: Feb. 22, 2024
(86) PCT No.: PCT/US2024/016845

§ 371 Date: Aug. 12, 2025

Related U.S. Application Data
(60) Provisional application No. 63/486,418, filed on Feb. 22, 2023.

(51) Int. Cl. A61B 17/11; A61L 31/04; A61L 31/06; A61L 31/14; A61L 31/16

(52) U.S. Cl. CPC A61B 17/1128; A61L 31/042; A61L 31/06; A61L 31/145; A61L 31/16

(57) ABSTRACT

Nanofiber nerve caps, methods of their preparation, and methods of their use for inhibiting neuroma formation associated with peripheral nerve injury are disclosed.

### CROSS-REFERENCE TO RELATED APPLICATIONS

This application claims the benefit of U.S. Provisional Application No. 63/486,418, filed Feb. 22, 2023, which is incorporated herein by reference in its entirety.

### FEDERALLY SPONSORED RESEARCH OR DEVELOPMENT

This invention was made with government support under grant W81XWH-21-1-0172 awarded by the United States Army Medical Research and Development Command. The government has certain rights in the invention.

### TECHNICAL FIELD

The presently disclosed subject matter generally relates to nanofiber nerve caps, methods of their preparation, and methods of their use for inhibiting neuroma formation associated with peripheral nerve injury.

### BACKGROUND

Peripheral nerve injuries are common and can result from a vast array of mechanisms, including trauma and iatrogenic surgical injury. When possible, the goals are to repair the injured nerves and facilitate reinnervation of target tissues, such as muscle or skin. Although these goals are feasible with small and distal nerve injuries, it is often not possible to achieve these goals with proximal nerve injuries due to the long distances between regenerating nerves and target tissues. Höke, 2006.

Whenever a nerve cannot be repaired and reunited with an end target, as in the case of limb amputation, the free nerve endings regenerating from the proximal nerve stump form aggregates of disorganized neural growth known as neuromas. Williams, 1984; Lewin-Kowalik, 2006. These neuromas exhibit spontaneous activity and pain, which is further exacerbated by mechanical or chemical stimuli. Cattell and Ville, 1961. The exact mechanisms that lead to experience of pain are not fully understood, but failed regenerative attempts with excessive and undirected growth of the injured axons play an important role in generating the pain.

There have been many advances in understanding why nerve regeneration fails in humans, especially with long distance regeneration. Höke, 2006; Scheib and Höke, 2013. Further, many of the molecular mechanisms that underlie failed regeneration of axons in the central nervous system (CNS) are known. Chew et al., 2012; Cregg et al., 2014. These molecular mechanisms that inhibit axon regeneration can potentially be used to prevent aborted growth at the distal end of a cut nerve and prevent neuroma formation, thereby potentially preventing neuroma pain or stump pain.

Many treatments have been proposed for treating painful neuromas, all of which, however, suffer from limitations.

### SUMMARY

In some aspects, the presently disclosed subject matter provides a nerve cap for inhibiting neuroma formation and/or growth at a site of nerve injury, wherein the nerve cap a has tubular body having an open aperture proximate a severed end of the nerve and a cap distal the severed end of the nerve, wherein the tubular body has a shape defined by an outer wall, wherein an inner surface of the outer wall further defines a lumen, and wherein the tubular body is configured to cover the severed end of the nerve at the site of injury.

In certain aspects, the outer wall comprises a nanofiber matrix comprising one or more bioactive compounds.

In some aspects, the presently disclosed subject matter provides a method for preparing a nerve cap having a predetermined shape, the method comprising: (a) preparing or providing an outer nanofiber scaffold; (b) deposing a portion of the outer nanofiber scaffold over a mold having a predetermined shape and having a tip of a predetermined shape; (c) heating the outer nanofiber scaffold at a temperature sufficient to at least partially melt fibers of the outer nanofiber scaffold while the scaffold is deposed on the mold; and (d) allowing the heated outer nanofiber scaffold to solidify to form the nerve cap.

In some aspects, the presently disclosed subject matter provides a method for inhibiting axon regeneration, promoting growth conc collapse, and/or preventing or inhibiting neuroma formation associated with a nerve injury, the method comprising capping an injured nerve of the subject with a presently disclosed nerve cap.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1A is a schematic view of an application of the presently disclosed nerve cap 100, showing injured nerve 200 and severed end of injured nerve 210;

FIG. 1B is a schematic view of the presently disclosed nerve cap 100;

FIG. 2A and FIG. 2B show one embodiment of the fabrication of the outer electrospun nerve cap. (FIG. 2A) A schematic depicting one embodiment strategy of an electrospun PCL nerve cap. (FIG. 2B) A positive nerve cap mold with an about 1.60-mm diameter and a hemispherical tip designed for a cap about 9 mm in length;

FIG. 3A, FIG. 3B, and FIG. 3C show representative nerve cap compositions and one embodiment. (FIG. 3A) A schematic depicting many possible formulations of nerve cap devices arising from variability and combinations thereof in the nanofiber fabrication, bioactive nanofiber conjugation, and filler hydrogel composition. (FIG. 3B) One nerve cap embodiment using an outer electrospun PCL nanofiber cap with an inner IPN consisting of NHC+CSPG. (FIG. 3C) One embodiment of a PCL outer cap+NHC/CSPG inner gel in capping a severed rat sciatic nerve stump;

FIG. 4 is a plot of the localized neuroma pain score versus time with the presently disclosed nerve cap;

FIG. 5 shows eliciting Tinel's sign to assess pain at coaptation site. By week 23, behavioral responses to mechanical stimulation of the coaptation site were significantly lower in the Conduit+CSPG animals as compared to Conduit (p<0.01), Direct Repair (p<0.001), and Neuroma groups (p<0.0001), demonstrating successful prevention of neuroma formation at the coaptation site. This decline in pain responses in the CSPG-Conduit group is particularly seen after Week 12 when the majority of nerves have fully regenerated and the pain inherent to regeneration is diminished. In contrast, the Neuroma group pain scores continue to increase beyond Week 12, nearing the maximal pain score of 20. Direct Repair animals consistently exhibited greater pain responses than Conduit+CSPG animals even in the early regeneration period between Weeks 4-10, demonstrating the utility of the Conduit+CSPG in comparison to nerve repair alone. The positive control (sham surgery) group consistently retains low response scores throughout the testing period and pain scores between the Conduit+CSPG and positive control (sham surgery) are no longer statistically significant at Week 23 (p=0.1347). Conduit and Direct Repair groups exhibited similar pain responses throughout the testing period. At Week 23, Conduit group pain scores were significantly lower than Neuroma scores (p<0.01), indicating benefit in using the conduit alone. Statistical analysis was performed using an ordinary one-way ANOVA with Tukey's post hoc test. Bars represent mean±SEM (ns=not significant, **p<0.01, ****p<0.0001). At week 23, sample sizes (n) are 6, 7, 9, 10, and 4 for Neuroma, Direct Repair, Conduit, Conduit+CSPG, and Positive Control groups, respectively; and

FIG. 6A, FIG. 6B, and FIG. 6C show axonal regeneration across coaptation site. (FIG. 6A-FIG. 6B) By week 23, maximal regeneration across the coaptation site had occurred. Assessment of neuromuscular junction reinnervation in the target muscle and retrograde-labelled cell bodies in the spinal cord demonstrated no significant differences across conduit and direct repair groups, demonstrating successful target reinnervation. (FIG. 6C) Tapered axonal growth was observed in Conduit+CSPG animals through staining with neurofilament (blue). Statistical analysis was performed using an ordinary one-way ANOVA with Tukey's post hoc test. Bars represent mean±SD.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

The presently disclosed subject matter provides nerve caps that can be used to prevent painful neuroma growth in peripheral nerve injury patients, with a particular focus on amputee patients. In some embodiments, the presently disclosed nerve cap can cap the end of a severed nerve in a patient to prevent unwanted nerve growth through a physical dominant mechanism and/or a biochemical dominant mechanism. The presently disclosed nerve caps differ from nerve caps and cuffs known in the art, at least in part, through the inclusion of a hydrogel-based filler in the lumen, as well as, in some embodiments, the encapsulation/conjugation of substances, such as bioactive agents and/or cells in the nanofiber matrix that influence nerve growth.

### A. Nerve Cap for Inhibiting Neuroma Formation and/or Growth at a Site of Nerve Injury

Accordingly, in some embodiments, the presently disclosed subject matter provides a nerve cap for inhibiting neuroma formation and/or growth at a site of nerve injury, wherein the nerve cap has a tubular body having an open aperture proximate a severed end of the nerve and a cap distal the severed end of the nerve, wherein the tubular body has a shape defined by an outer wall, wherein an inner surface of the outer wall further defines a lumen, and wherein the tubular body is configured to cover the severed end of the nerve at the site of injury.

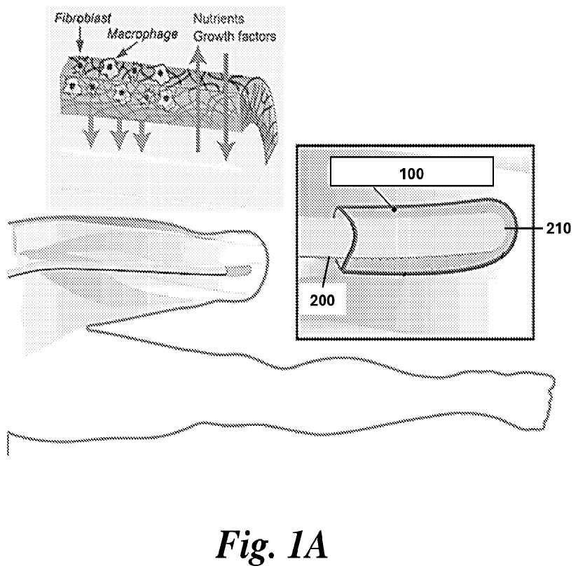

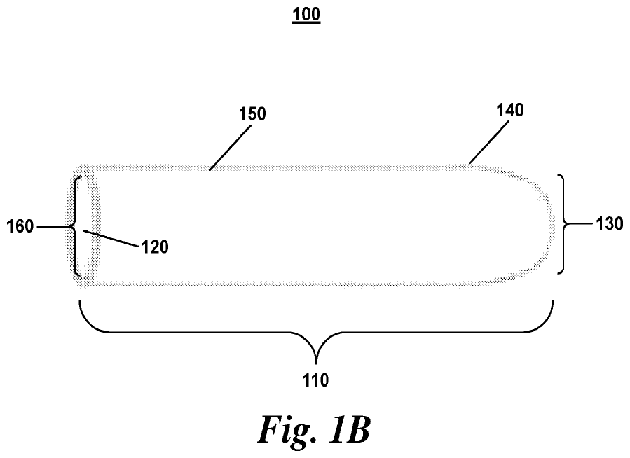

Referring now to FIG. 1A, shown is nerve cap 100 configured to cover a severed end 210 of injured nerve 200. Referring now to FIG. 1B is nerve cap 100. Nerve cap 100 includes tubular body 110 having an open aperture 120 and cap 130. Tubular body 110 further comprises outer wall 140. Outer wall 140 further comprises an inner surface 140, which defines lumen 160.

In some embodiments, the outer wall comprises a nanofiber matrix comprising one or more bioactive compounds.

In certain embodiments, the nanofiber matrix comprises a plurality of nanofibers selected from electrospun nanofibers, including multi-jet electrospun nanofibers, core/shell nanofibers, layer-by-layer nanofibers, emulsion nanofibers, and combinations thereof. In certain embodiments, the nanofiber matrix comprises a semi-permeable matrix which allows for nutrient and fluid transport and immunomodulation via macrophage entrapment.

In particular embodiments, the nanofiber matrix has one or more characteristics selected from: (a) a thickness ranging from about 50 μm to about 500 μm, including about 50, 60, 70, 80, 90 100, 200, 300, 400, and 500 μm; (b) a pore size of less than about 10 μm, including about 9, 8, 7, 6, 5, 4, 3, 2, and 1 μm; (c) a fiber diameter ranging from about 100 nm to about 2 μm, including about 100, 200, 300, 400, 500, 600, 700, 800, 900, 1000, 1100, 1200, 1300, 1400, 1500, 1600, 1700, 1800, 1900, and 2000 nm; and (d) a plurality of randomly oriented nanofibers.

In certain embodiments, the nanofiber matrix comprises one or more synthetic materials selected from poly(ε-caprolactone) (PCL), copolymers of ε-caprolactam and hexamethylendiaminadipate, polyglycolic acid (PGA), poly(lactic acid) (PLA), poly (1-lactic acid) (PLLA), copolymers of PLA and PGA, poly(lactic-co-glycolic acid) (PLGA), poly(vinyl acetate) (PVA), poly(ethylene-co-vinyl acetate) (PEVA), poly(ethylene glycol) (PEG), polyurethanes (PU), poly(ethylene oxide) (PEO), poly(vinyl pyrrolidone) (PVP), poly(ethylene terephthalate) (PET), poly(glycerol sebacate) (PGS), polydioxanone (PDO), polyphosphazenes (PPHOs), polyhydroxyalkanoates (PHA), polyhydroxybutyrates (PHB), polyhydroxyvalerate (PHV), polyhydroxyhexanoate (PHH), polyhydroxyoctanoate (PHO), and co-polymers, blends, analogs, derivatives, modifications, and mixtures thereof; and/or one or more natural materials selected from hyaluronic acid (HA), silk, keratin, collagen, gelatin, fibrinogen, elastin, actin, myosin, cellulose, amylose, dextran, chitin, glycosaminoglycans (GAG), deoxyribonucleic acids (DNA), ribonucleic acids (RNA), chitin, chitosan (CS), alginate, and co-polymers, blends, analogs, derivatives, modifications, and mixtures thereof.

### A.1 Nerve Caps Comprising Core-Shell Structure Nanofibers

In certain embodiments, the nanofiber matrix comprises a plurality of core-shell structure nanofibers or a multilayered nanofiber assembly. In particular embodiments, the one or more bioactive molecules are encapsulated in the core-shell structure nanofibers or multilayered nanofiber assembly, e.g., for controlled release.

In certain embodiments, the plurality of core-shell structures or multilayered nanofiber assembly are located at a predetermined location in the nanofiber matrix or are dispersed throughout the nanofiber matrix. In particular embodiments, the plurality of core-shell structures are located in the nanofiber matrix at the cap of the tubular body distal the severed end of the nerve.

### A.2 Nerve Caps Comprising a Hydrogel-Based Filler or a Semi-Interpenetrating Network (IPN) Filler

In some embodiments, the lumen is open. In other embodiments, the lumen comprises a hydrogel-based filler or a semi-interpenetrating network (IPN) filler to further inhibit axon regeneration. Accordingly, in some embodiments, the lumen can be substantially open, i.e., between about 100% to about 95% open, including about 100% open, 99.9% open, 99.5% open, 99% open, 98% open, 97%, 96% open, and 95% open. In other embodiments, the lumen can be partially filled, e.g., between about 0.5% to less than about 95% filled, including about 0.5%, 1%, 5%, 10%, 15%, 20%, 25%, 30%, 35%, 40%, 45%, 50%, 55%, 60%, 65%, 70%, 75%, 80%, 85%, 90%, and less than 95% filled. In other embodiments, the tip of the lumen comprises the hydrogel-based filler or IPN filler. In yet other embodiments, the hydrogel-based filler or IPN filler can be applied to an interior or inside wall of the lumen.

Methods for preparing hydrogels suitable for use with the presently disclosed nerve caps are disclosed in International PCT Patent Application Publication No. WO 2022/081856 A1 for Biodegradable Nanofiber Conical Conduits for Nerve Repair, to Mao et al., published Apr. 21, 2022, which is incorporated herein by reference in its entirety.

The term “lumen” is used interchangeably with the term “channel,” “inner space,” “cavity,” and the like to describe an inner volume of the nerve cap tubular body that can be filled with a hydrogel.

In certain embodiments, the hydrogel filler comprises an architecture selected from a nanofiber-hydrogel composite (NHC), an NHC interpenetrating network, an NHC comprising conjugated nanofiber fragments, and a loaded hydrogel.

In certain embodiments, the NHC comprises functionalized poly(ε-caprolactone) (PCL) fiber fragments distributed in and covalently conjugated to a hydrogel network formed by reacting acrylated hyaluronic acid (HA) with thiolated poly(ethylene glycol) (PEG-SH).

In certain embodiments, the hydrogel-based material or semi-interpenetrating network (IPN) filler inhibits axonal growth, polarizes macrophages to the pro-regenerative phenotype, supports angiogenesis, and combinations thereof.

In certain embodiments, the hydrogel-based material further comprises one or more bioactive agents covalently or non-covalently bond thereto.

In particular embodiments, the hydrogel-based material is selected from methacrylated hyaluronic acid (MeHA) crosslinked with thiolated poly(ethylene glycol) (PEG-SH), MeHA crosslinked with PEG-SH and chondroitin sulfate proteoglycans (CSPGs), MeHA cross-linked with PEG-SH, CSPGs, and PCL nanofiber fragments, a material that resembles native ECM, and combinations thereof, wherein the PEG-SH can be substituted by other chemical cross-linkers. In certain embodiments, the hydrogel-based material is selected from a fibrin-, a collagen-, or a tissue matrix-derived hydrogel.

In particular embodiments, the hydrogel-based material comprises MeHA and CSPGs, and wherein the MeHA acts as a structural hydrogel that mimics native extracellular matrix (ECM), thereby resembling an environment of a peripheral nerve, and wherein the CSPGs inhibit axonal growth for preventing neuroma formation.

In particular embodiments, the hydrogel-based material comprises crosslinked PCL nanofiber fragments with hyaluronic acid (creating a nanofiber-hydrogel composite (NHC) that promotes macrophages polarization to the pro-regeneration phenotype and angiogenesis).

In particular embodiments, the hydrogel-based material is physically mixed with CSPG to create a semi-interpenetrating network.

In certain embodiments, the hydrogel-based material has an overall stiffness (shear storage modulus, G′) ranging from about 50 Pa to about 500 Pa, including about 50, 60, 70, 80, 90, 100, 200, 300, 400, and 500 Pa.

### A.3 Nerve Caps Comprising One or More Bioactive Molecules

In some embodiments, the one or more bioactive molecules comprising the nanofiber matrix and/or the hydrogel-based filler or a semi-interpenetrating network filler recapitulate membrane bound signaling molecules.

In some embodiments, the one or more bioactive compounds comprising the nanofiber matrix and/or the hydrogel-based filler or a semi-interpenetrating network filler comprise one or more extracellular matrix components.

In some embodiments, the one or more bioactive agents comprising the nanofiber matrix and/or the hydrogel-based filler or a semi-interpenetrating network filler inhibit axonal outgrowth.

In some embodiments, the one or more bioactive agents that inhibit axonal outgrowth are selected from a semaphorin, a myelin-associated glycoprotein, and one or more chondroitin sulfate proteoglycans (CSPGs).

In some embodiments, the one or more bioactive molecules comprising the nanofiber matrix and/or the hydrogel-based filler or a semi-interpenetrating network filler are selected from a small molecule, a chondroitin sulfate proteoglycan (CSPG), a bioactive protein, an intracellular signaling pathway involved in growth cone collapse, such as a cyclic nucleotide (for continuous inhibition of nerve growth), a nucleic acid, a bioactive molecule-secreting cell, a stem cell, an hiPSC-derived Schwann Cell (continuously secrete aggrecans (a form of CSPG) and/or semaphorins), and combinations thereof.

In some embodiments, the secreted protein comprises a semaphorin that provides axon guidance during development by inhibiting growth cones.

In some embodiments, the one or more bioactive compounds are covalently bound to nanofibers of the nanofiber matrix and/or the hydrogel-based filler or a semi-interpenetrating network filler (to slowly release one or more small molecules that inhibit axon regeneration).

In some embodiments, the nanofibers of the nanofiber matrix comprise PCL and the one or more bioactive compounds, e.g., CSPGs and semaphorins, are conjugated onto the PCL nanofibers via grafted poly-(acrylic acid) (PAA).

### A.4 Morphology and Other Characteristics of Presently Disclosed Nerve Caps

In some embodiments, the outer wall of the tubular body has one or more surfaces having a morphology selected from a smooth morphology, a crimped morphology, and combinations thereof.

In some embodiments, the crimped morphology has a kink-resistance of up to a 90° bend and a length adjustability of less than or equal to 100% of an initial cap length. In some embodiments, the nerve cap further comprises one or more suturable extensions, including a proximate extension.

In some embodiments, the one or more extensions has one or more dimensions ranging in diameter from about 50 μm to about 25 mm, including about 0.05, 0.5, 1, 2, 3, 4, 5, 6, 7, 8, 9, 10, 11, 12, 13, 14, 15, 16, 17, 18, 19, 20, 21, 22, 23, 24, and 25 mm and in length from about 0 mm to about 50 mm, including about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mm;

In some embodiments, the nerve cap has one or more characteristics selected from:
(a) an open proximate end having a diameter ranging from about 0 mm to about 25 mm, including 0.1, 0.5. 1, 2, 3, 4, 5, 6, 7, 8, 9, 10, 11, 12, 13, 14, 15, 16, 17, 18, 19, 20, 21, 22, 23, 24, and 25 mm;
(b) a closed distal end having a shape selected from rounded (e.g., a cap), pointed (e.g., conical), or cubic (e.g., a prism);
(c) a uniform diameter along a length of the cap or a gradient diameter along a length of the cap (e.g., a cone); and
(d) a length of the cap ranging from about 5 mm to about 50 mm, including about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mm.

### B. Methods for Preparing Nerve Caps

In some embodiments, the presently disclosed subject matter provides a method for preparing a nerve cap having a predetermined shape, the method comprising:
(a) preparing or providing an outer nanofiber scaffold;
(b) deposing a portion of the outer nanofiber scaffold over a mold having a predetermined shape and having a tip of a predetermined shape;
(c) heating the outer nanofiber scaffold at a temperature sufficient to at least partially melt fibers of the outer nanofiber scaffold while the scaffold is deposed on the mold; and
(d) allowing the heated outer nanofiber scaffold to solidify to form the nerve cap.

In some embodiments, the mold comprises a three-dimensional cylindrical mold.

In some embodiments, the tip of the mold has a predetermined shape selected from a rounded tip (e.g., a cap), a pointed tip (e.g., conical), and a cubic tip (e.g., a prism).

In some embodiments, the outer nanofiber scaffold is prepared by one or more methods selected from electrospinning a polymer solution, rotary jet spinning of a polymer solution, phase separation of a polymer solution, polymer nanofiber self-assembly, magnetospinning a polymer solution, and melt blowing a polymer melt.

In some embodiments, the electrospinning is conducted under one or more conditions selected from:
(a) a concentration of the polymer solution ranging from about 5 wt % to about 20 wt % of polymer, including about 5, 6, 7, 8, 9, 10, 11, 12, 13, 14, 15, 16, 17, 18, 19, and 20 wt %;
(b) a molecular weight of the polymer ranging from 15,000 to about 100,000, including about 15000, 20000, 25000, 30000, 35000, 40000, 45000, 50000, 55000, 60000, 65000, 70000, 75000, 80000, 85000, 90000, 95000, and 100000;
(c) an electrospinning voltage having a range from about 5 kV to about 24 kV, including about 5, 6, 7, 8, 9, 10, 11, 12, 13, 14, 15, 16, 17, 18, 19, 20, 21, 22, 23, and 24 kV
(d) a distance from a polymer solution nozzle, needle, or spinneret to a rotating mandrel having a range from about 3 cm to about 20 cm, including about 3, 4, 5, 6, 7, 8, 9, 10, 11, 12, 13, 14, 15, 16, 17, 18, 19, and 20 cm; and
(e) a mandrel rotation speed having a range from about 50 RPM to about 1000 RPM, including about 50, 100, 200, 300, 400, 500, 600, 700, 800, 900, and 1000 RPM.

In some embodiments, the polymer comprises one or more synthetic materials selected from poly(ε-caprolactone) (PCL), copolymers of ε-caprolactam and hexamethylendiaminadipate, polyglycolic acid (PGA), poly(lactic acid) (PLA), poly (1-lactic acid) (PLLA), copolymers of PLA and PGA, poly(lactic-co-glycolic acid) (PLGA), poly(vinyl acetate) (PVA), poly(ethylene-co-vinyl acetate) (PEVA), poly(ethylene glycol) (PEG), polyurethanes (PU), poly(ethylene oxide) (PEO), poly(vinyl pyrrolidone) (PVP), poly(ethylene terephthalate) (PET), poly(glycerol sebacate) (PGS), polydioxanone (PDO), polyphosphazenes (PPHOs), polyhydroxyalkanoates (PHA), polyhydroxybutyrates (PHB), polyhydroxyvalerate (PHV), polyhydroxyhexanoate (PHH), polyhydroxyoctanoate (PHO), and co-polymers, blends, analogs, derivatives, modifications, and mixtures thereof.

In some embodiments, the polymer is dissolved in an organic solvent selected from ethyl ether, hexane, tetrachloroethane, toluene, xylene, hexafluoro-2-propanol, and analogs, derivatives, modifications, and mixtures thereof.

In some embodiments, the method comprises electrospinning an about 8-wt % PCL solution using 80,000 molecular weight (MW) PCL in a 9:1 solvent of dichloromethane (DCM) and dimethylformamide (DMF) at about 10 kV at a 10-cm distance from a mandrel rotating at about 140 RPM.

In some embodiments, the method further comprises functionalizing nanofibers of the outer nanofiber scaffold with one or more bioactive molecules.

In some embodiments, the one or more bioactive molecules are conjugated to the nanofiber.

In some embodiments, the one or more bioactive molecules are conjugated to the nanofiber through grafting.

In some embodiments, the grafting includes direct copolymerization, end functionalization, bulk grafting, solution grafting, use of an emulsion or mini-emulsion technique, use of supercritical conditions, gamma irradiation, electron beam, visible light exposure, ultraviolet irradiation, and mechanical shear.

In some embodiments, the conjugation is introduced by the use of one or more of an enzyme, an alkene, an alkoxyamine, a ketone, a thiol, a norbornene, a propargyl, an alkyne, an azide, a carboxylic acid, and combinations, analogues, or derivatives thereof.

In some embodiments, the method further comprises removing excess material beyond a sealed end of the nerve cap.

In some embodiments, the method further comprises forming extensions or alterations to a surface morphology of the nerve cap by one or more of:
(a) electrospinning directly onto a mold with the desired structure;
(b) fabricating electrospun sheets to cover a mold;
(c) introducing a crimped surface morphology and associated techniques therein;
(d) using a negative mold or negative and positive mold combination for shaping; and
(e) changing the size or shape of the nerve cap mold.

In some embodiments, the method further comprises at least partially filling a lumen of the nerve cap with a hydrogel-based filler material or a semi-interpenetrating network (IPN) filler, wherein the hydrogel-based filler material or a semi-interpenetrating network (IPN) filler can further comprise one or more bioactive compounds.

In some embodiments, the at least partially filing of the lumen of the nerve cap is done during manufacture of the nerve cap. In other embodiments, the at least partially filing of the lumen of the nerve cap is done at the time of implant. Different fillers and nerve caps can be used for different indications, based on, for example, the size of the nerve, the location of the nerve, the primary function of the nerve (e.g., sensory vs motor), and the like.

C. Methods for Inhibiting Axon Regeneration, Promoting Growth Cone Collapse, and/or Preventing or Inhibiting Neuroma Formation Associated With a Nerve Injury

In some embodiments, the presently disclosed subject matter provides a method for inhibiting axon regeneration, promoting growth conc collapse, and/or preventing or inhibiting neuroma formation associated with a nerve injury, the method comprising capping an injured nerve of the subject with a presently disclosed nerve cap.

In some embodiments, the closed distal end of the nerve cap provides a physical barrier to prevent or inhibit continued nerve growth.

In some embodiments, the nerve injury comprises a peripheral nerve injury.

In some embodiments, the nerve injury arises as a result of an amputation or another case of significant nerve injury.

In some embodiments, the subject is an amputee.

As used herein, the presently disclosed nerve caps can be used to mitigate, minimize, or prevent formation of neuromas in severed, including divided or sectioned, or damaged nerve endings, in particular, peripheral nerve endings, including managing pain associated with severed or damaged nerve endings.

The terms “minimizing” or “mitigating” are intended to mean that the use of the presently disclosed nerve conduit on a nerve ending substantially reduces pain and severity of any symptoms associated with neuromas, i.e., clusters of disorganized nerve fibers, while not necessarily completely preventing or inhibiting formation of a neuroma over time. While some disorganized neural growth may still occur over time, the use of a presently disclosed nerve conduit in accordance with certain aspects of the present disclosure advantageously reduces symptoms and pain as compared to conventional neuroma treatment techniques known in the art.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or discase or the prophylactic treatment for preventing the onset of a condition or discase, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Nanofiber Nerve Caps to Inhibit Neuroma Formation

### 1.1 Background

One of the first proposed treatments for neuroma formation was simple excision of the neuroma, often with concurrent purposeful damage the proximal nerve end to attempt to reduce regeneration, such as in a traction neurectomy. Dellon et al., 1984.This form of treatment usually results in immediate reduction in pain, but is limited by the eventual recurrence of the neuroma and its associated symptoms. More recently, long pieces of nerve allograft have been used following excision of the neuroma as axonal regeneration slowly “fizzles out” along the length of the graft. This procedure prevents the formation of one large neuroma. Nerves treated this way, however, can still be sensitive. Eberlin and Ducic, 2018.

A number of surgical approaches have been developed to treat symptomatic neuroma pain and/or prevent neuroma formation. Each procedure has demonstrated partial alleviation of the pain; however, all of the procedures have significant drawbacks that limit the effective prevention of neuroma formation. As many neuromas are superficially located under the skin and easily stimulated, excision and relocation to a quiescent environment, such as in muscle or bone, has been a mainstay of treatment since the 1980s. Eberlin and Ducic, 2018; Dumanian et al., 2019. This technique provides modest benefits, presumably from protecting the neuroma stump from direct mechanical stimulation. When an injured nerve is buried into innervated muscle, however, a neuroma will form within the substance of the muscle because innervated muscle will not accept additional innervation from the buried nerve. Frey et al., 1982; Guth and Zalewski, 1963.

Currently, the most promising approach to treat perineural invasion (PNI) is targeted muscle reinnervation (TMR), a procedure that involves suturing the injured proximal nerve stump to a distal nerve target in a freshly cut motor nerve. This procedure allows the axons regenerating from the proximal nerve stump to innervate a distal target that also provides a muscle interface for the severed nerve. Dumanian et al., 2019; Cheesborough et al., 2015. The major limitation inherent to TMR resulting in treatment failure is the substantial size-mismatch between the large caliber proximal nerve stump and the much smaller distal nerve branch to which it is connected, which results in size-mismatches on a scale of 5-to-1 or greater. Consequently, many of the regenerating axons from the proximal nerve stump that escape the repair site form a neuroma.

Few devices have been developed as surgical adjuncts to address neuromas. These devices primarily consist of nerve caps that attempt to block nerve growth and subsequent neuroma formation. Bushman, 2021. The efficacy of nerve caps, however, has been called into question, as it is difficult, if not impossible, to completely inhibit the process of regeneration through the nerve cap's physical inhibition. Previous research also has investigated the use of silicone tubes to prevent inflammatory cell infiltration after peripheral nerve injury. Okuda et al., 2006. These silicone tubes, however, are still insufficient for nerve repair for extended nerve defects and do not completely prevent potential neuroma formation and pain, thereby limiting their efficacy and adoption. Dahlin and Lundborg, 2001; Swanson et al., 1977. Straight nerve caps are currently available to bridge a defect within an injured nerve and thereby facilitate peripheral nerve regeneration and functional recovery. Yan et al., 2014; Nan et al., 2012. These straight conduits, however, are not useful in preventing neuroma formation.

### 1.2 Overview

The presently disclosed subject matter provides a combination of nanofiber matrix and molecular strategies that inhibit axon regeneration and promote growth cone collapse to prevent neuroma formation. The strategies that will be employed in inhibiting regeneration will include extracellular matrix components, such as chondroitin sulfate proteoglycans (CSPGs), Höke and Silver, 1996; Sandvig et al., 2004, secreted proteins, such as semaphorins that provide axon guidance during development by inhibiting growth cones, Derijck et al., 2010, and intracellular signaling pathways involved in growth cone collapse, such as cyclic nucleotides. Bashaw and Klein, 2010.

### 1.3 Design Features and Function of Presently Disclosed Nerve Cap

The presently disclosed nerve cap for neuroma inhibition has the following design features including, but not limited to: (a) electrospun nanofiber matrix nerve cap to cover the severed end of a nerve at the site of injury; (b) a semi-permeable matrix to allow for nutrient and fluid transport and immunomodulation via macrophage entrapment; (c) functionalization of nanofibers with covalently bound bioactive compounds including, but not limited to CSPGs, semaphorins, cyclic nucleotides, and the like, and slow release of small molecules that inhibit axon regeneration; (d) encapsulation of bioactive molecules/bioactive molecule-secreting cells in core shell structure nanofibers; and (e) a hydrogel-based bioactive material as a filler to further inhibit axon regeneration.

### 1.4 Semi-Permeable Electrospun Nanofiber Matrix Cap

A suturable nerve cap addresses the issue of neuroma at severed ends of peripheral nerves by serving as a physical barrier to prevent nerve growth, particularly in amputee patients where there are cases of significant nerve injury. The cap is characterized by its cover-like form where one end is open and the other is closed. In between the two ends, the cap may be of uniform diameter (as seen in FIG. 1) or of a gradient diameter (i.e., a cone). The diameter of the open-end ranges from about 0 mm to about 25 mm to accommodate nerves of various sizes. As the opposite end of the cap is closed, no diameter applies; however, it may come in a variety of shapes including rounded (as seen in FIG. 1), pointed (conical), or cubic (prism). The length of the cap ranges from about 5 mm to about 50 mm. In one embodiment, the length of the cap is about 5 mm and the form appears as a cap.

The wall of the cap can be either smooth or crimped in nature. The matrix of the cap can be disposed to form ridges (crimping) characterized by both kink-resistance of up to a 90° bend and length adjustability of less than or equal to 100% of the initial cap length. This configuration allows the cap to be tailored to various lengths as warranted. A longer cap also can be cut to remove excess length.

The nerve cap consists of a nanofiber matrix with a relatively uniform thickness ranging from about 50 to about 500 μm and a pore size of less than about 10 μm. The cap is composed of randomly oriented nanofibers with fiber diameter ranging from about 100 nm to about 2 μm in diameter. Previous findings suggest that these ranges of fiber diameters and pore sizes are ideal for entrapping and promoting macrophage polarization toward the M2 pro-regenerative phenotype, thereby reducing the inflammatory reaction towards the cap. Sarhane et al., 2019. Further, the porosity of the cap allows for adequate nutrient diffusion through the cap wall.

The nanofiber matrix may be composed of a range of acceptable synthetic and natural polymers for medical applications, including their composites. Preferred synthetic materials for the matrix include, but are not limited to, the polymers poly(ε-caprolactone) (PCL), copolymers of s-caprolactam and hexamethylendiaminadipate, polyglycolic acid (PGA), poly(lactic acid) (PLA), poly (1-lactic acid) (PLLA), copolymers of PLA and PGA, poly(lactic-co-glycolic acid) (PLGA), poly(vinyl acetate) (PVA), poly(ethylene-co-vinyl acetate) (PEVA), poly(ethylene glycol) (PEG), polyurethanes (PU), poly(ethylene oxide) (PEO), poly(vinyl pyrrolidone) (PVP), poly(ethylene terephthalate) (PET), poly(glycerol sebacate) (PGS), polydioxanone (PDO), polyphosphazenes (PPHOs), polyhydroxyalkanoates (PHA), polyhydroxybutyrates (PHB), polyhydroxyvalerate (PHV), polyhydroxyhexanoate (PHH), and polyhydroxyoctanoate (PHO), as well as co-polymers, blends, analogs, derivatives, modifications, and mixtures thereof. Ogle et al., 2014; Al-Enizi et al., 2018.

Other suitable natural materials for the matrix include, but are not limited to, the polymers hyaluronic acid (HA), silk, keratin, collagen, gelatin, fibrinogen, elastin, actin, myosin, cellulose, amylose, dextran, chitin, glycosaminoglycans (GAG), deoxyribonucleic acids (DNA), ribonucleic acids (RNA), chitin, chitosan (CS), alginate, as well as co-polymers, blends, analogs, derivatives, modifications, and mixtures thereof. Al-Enizi et al., 2018; Painter and Coleman, 2008.

### 1.5 Bioactive Molecule-Conjugation in Nanofiber Matrix

Electrospun nanofiber matrices may be used to deliver bioactive proteins, small molecules, or encapsulated stem cells. These conjugated nanofibers are amenable to highly modular kinetic release of the bioactive components. Additional covalent attachment of biologically active proteins to the nanofiber structure can be achieved to recapitulate membrane bound signaling molecules, which have previously been shown to alter biological properties of Schwann cells and axons in the peripheral nervous system. Bockelmann et al., 2011.

In one embodiment, CSPGs and/or semaphorins are conjugated onto PCL nanofibers via grafted PAA and subsequent incubation with CSPGs and semaphorins. These bioactive compounds are known to have a role in inhibiting axonal growth and thereby aid in preventing the excess neural growth characteristic of neuromas.

### 1.6 Core-Shell Structured Nanofibers for Cell or Molecule Encapsulation

Within the nanofiber matrix, core-shell structured nanofibers or other multi-layered nanofiber assemblies may be used for encapsulation and controlled release of bioactive molecules. These core-shell structures may be located at any point within the matrix or be present throughout the entire matrix. In one embodiment, cyclic nucleotides arc encapsulated in the core-shell structure at the closed end of the cap, where they achieve a sustained release profile for continuous inhibition of nerve growth. In another embodiment, hiPSC-derived Schwann Cells are encapsulated in the core-shell structure at the closed end of the cap, where they continuously secrete aggrecans (a form of CSPG) and/or semaphorins.

### 1.7 Hydrogel-Based Filler Material

The nerve cap's lumen may be empty or be filled with a hydrogel-based material. The gel filling of the cap aims to inhibit axonal growth, polarize macrophages to the pro-regenerative phenotype, and support angiogenesis, or any combination of these functions. Exemplary formulations for the gel filling include, but are not limited to, methacrylated hyaluronic acid (MeHA) crosslinked with thiolated poly(ethylene glycol) (PEG-SH), MeHA crosslinked with PEG-SH and chondroitin sulfate proteoglycans (CSPGs), and MeHA cross-linked with PEG-SH, CSPGs, and PCL nanofiber fragments. In certain embodiments, the gel can have an overall stiffness (shear storage modulus, G′) ranging from about 50 Pa to about 500 Pa. Li et al., 2020.

MeHA acts as a structural hydrogel that mimics native extracellular matrix (ECM), resembling the environment of the peripheral nerve and CSPGs inhibit axonal growth for preventing neuroma formation. Crosslinking of the PCL nanofiber fragments with hyaluronic acid has been shown to create a nanofiber-hydrogel composite (NHC) that promotes macrophages polarization to the pro-regeneration phenotype and angiogenesis. Sarhane et al., 2019.

The gel filling includes, but is not limited to, the materials provided hereinabove and any other co-polymers, blends, analogs, derivatives, modifications, and mixtures thereof. Substitutes for CSPGs can include other molecules that similarly inhibit axonal growth and prevent neuroma formation. The NHC can be synthesized with hydrogels other than hyaluronic acid that resemble native ECM, such as collagen and fibrin. PEG-SH can be substituted by other chemical cross-linkers.

In one embodiment, the nerve cap is filled with a nanofiber hydrogel composite physically mixed with CSPG to create a semi-interpenetrating network.

### Example 2

### Process of Manufacture

### 2.1 Fabrication of Outer Electrospun Nanofiber Cap

One strategy to fabricate the nanofiber matrix cap manufacturing process involves electrospinning a poly(ε-caprolactone) (PCL) polymer solution. In one embodiment, an about 8-wt % PCL solution using 80,000 molecular weight (MW) PCL in a 9:1 solvent of dichloromethane (DCM) and dimethylformamide (DMF), respectively is electrospun at about 10 kV at a 10-cm distance from a mandrel rotating at about 140 RPM. After electrospinning, the resulting nanofiber tube is sprayed with about 70% ethanol while on the mandrel to easily remove the fiber tube from the mandrel surface.

Manufacturing nanofibers of similar dimensions and properties can be accomplished through methods including, but not limited to, rotary jet spinning of polymer solutions, phase separation of polymer solutions, polymer nanofiber self-assembly, magnetospinning polymer solutions, and melt blowing a polymer melt. Additionally, the polymer solution can range from about 5 wt % to about 20 wt % with PCL MW ranging from about 15,000 to about 100,000. Alternatives to PCL and combinations thereof are listed hereinabove, and organic solvent alternatives can include other solvents capable of dissolving the polymer of choice, including but not limited to, ethyl ether, hexane, tetrachloroethane, toluene, xylene, hexafluoro-2-propanol, and analogs, derivatives, modifications, and mixtures thereof. Electrospinning voltages can range from about 5 kV to about 24 kV, with spin distances ranging from about 3 cm to about 20 cm and mandrel rotation speeds ranging from about 50 RPM to about 1000 RPM.

### 2.1.1. Bioactive Functionalization of Electrospun Nanofibers

A number of alternative embodiments to the fabrication of the outer electrospun nanofiber cap may include the use of functionalized nanofibers, or nanofibers conjugated to bioactive components for sustained delivery or display. The incorporation of bioactive proteins, glycoproteins, small molecules, nucleic acids and their derivatives, and biologicals via stem cell derivatives into the outer nanofiber matrix may be used to inhibit regeneration or prevent neuroma formation.

Two example alternative embodiments include the preparation of CSPG-or SEMA3A-conjugated nanofibers. PCL nanofibers may be grafted with poly-(acrylic acid) (PAA) via oxygen plasma treatment to introduce carboxylic groups. PAA-grafted nanofibers would then be incubated with 50 g/mL of CSPG or SEMA 3A solution in the presence of 5 mg/mL NHS (N-Hydroxysuccinimide) and EDC (N-(3-Dimethylaminopropyl)-N′-ethylcarbodiimide) at 4° C. overnight. The CSPG- or SEMA3A-conjugated nanofibers may then be washed with 0.01% Tween-20 in PBS (phosphate buffered saline) 3 times.

The use of PCL and PAA may be substituted for the polymers listed above. Modifications to introduce grafting may include, but are not limited to, direct copolymerization, end functionalization, bulk grafting, solution grafting, emulsion or miniemulsion techniques, use of supercritical conditions, gamma irradiation, electron beam, visible light exposure, ultraviolet irradiation, and mechanical shear. Similarly, strategies and chemistries to introduce conjugation may include, but are not limited to, the use of enzymes, alkenes, alkoxyamines, ketones, thiols, norbornenes, propargyls, alkynes, azides, and combinations, analogues, or derivatives thereof.

### 2.1.2 Fabrication of Core-Shell Nanofibers

Additional embodiments of nanofiber-mediated bioactive component encapsulation and sustained delivery may include the use of core-shell structured nanofibers or similar nanofiber structures, such as layer-by-layer nanofibers. One alternative embodiment involves the use of coaxial electrospinning to encapsulate and release cyclic nucleotides. In this respect, a coaxial nozzle may be used to electrospin a 8.5% PCL solution in a mixture solvent of chloroform and methanol (3:1, v/v) as an outer solution, and a 4.25 mg/mL of mixture of cyclic nucleotide and poly-(ethylene glycol) (PEG) may be prepared with 1% poly(vinyl alcohol) (PVA) in distilled water as an inner solution. Alternative polymer types, solvents, compositions, and strategies to prepare nanofibers are listed above.

### 2.2. Formation of Nerve Cap Shape

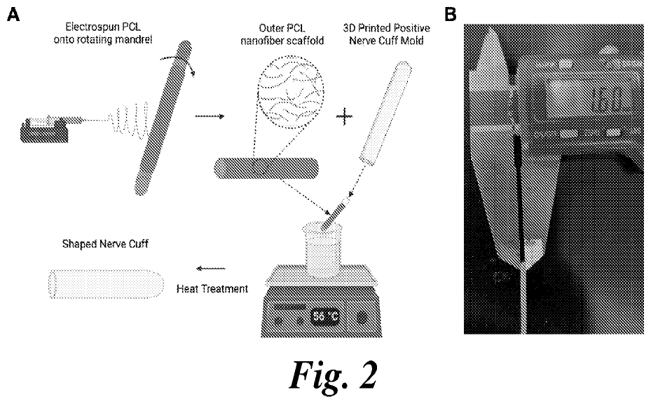

In one embodiment, the nerve cap is formed by cutting the electrospun PCL nanofiber scaffold tube into approximately 9.5-mm segments and stretching the tube over a three-dimensional cylindrical mold with a rounded tip at the end where the cap will be sealed off. Thereafter, the PCL scaffold is heat-treated in a heated water bath, e.g., about 56° C., for about 1 second to partially melt the PCL fibers, effectively increasing the mechanical properties of the conduit and solidifying the nerve cap structure by shaping it around the 3D-printed mold (FIG. 2). Excess material beyond the sealed end of the cap is then removed using a clean razor blade.

Alternative formulations of the nerve cap may include a hydrogel or semi-IPN filler. In these embodiments, suturable extensions may be incorporated by molding a portion of the PCL scaffold tube at the open end in excess to the cap length which may be filled with the hydrogel, thereby creating an appendage to the cap which can be fitted over the nerve stump for excess suturable area.

The formation of the nerve cap shape and its associated extensions or alterations to the surface morphology may be obtained through alternative methods, including but not limited to, electrospinning directly onto a mold with the desired structure, fabricating electrospun sheets to cover a mold, introducing a crimped surface morphology and associated techniques therein, using a negative mold or negative and positive mold combination for shaping, and changing the size or shape of the nerve cap mold.

### 2.3 Synthesis of Hydrogel-Based Filler Material

In one embodiment of the nerve cap, a hydrogel layer may be incorporated into the lumen of the cap. The hydrogel filling may be composed of a bulk hydrogel or a nanofiber hydrogel composite (NHC) alone or with a chondroitin sulfate proteoglycan (CSPG) network to form a semi-interpenetrating network (IPN). In one embodiment, the NHC is composed of methacrylated hyaluronic acid (HA), cryomilled functionalized PCL fiber fragments, and thiolated poly(ethylene glycol) (PEG-SH).

MeHA is either purchased directly from commercial manufacturers or synthesized from hyaluronic acid and glycidyl acrylate. In one embodiment, MeHA is produced by creating an about 1% w/v solution of HA in phosphate-buffered saline and adding about 3.25 mL of glycidyl acrylate to about 100 mL of about 1% HA solution. The entire solution is shaken at about 200 RPM at about 37° C. for about 16 hours. The solution is dripped into about 1 L of about 100% ethanol, and the precipitated MeHA is collected and washed with about 100% ethanol three times before drying in the hood. Synthesis methods for MeHA include, but are not limited, to this process.

In one embodiment, about 5000 MW PEG-SH is manufactured and purchased commercially. Synthesis methods, however, are available to create PEG-SH or alternate cross-linkers.

PCL fiber fragments are produced by electrospinning a PCL nanofiber sheet before surface functionalization and cryomilling. Al-Enizi et al., 2018. In one embodiment, parameters include spinning an about 16 wt % solution of PCL in an about 85:15 mixture of about 45,000:80,000 MW PCL dissolved in a about 9:1 organic solvent of DCM: DMF. Electrospinning parameters include spinning at about 16 kV at a distance of about 16 cm from a steel drum rotating at about 750 RPM. Following the electrospinning, the PCL sheet is cut into about 100 cm² sheets and plasma treated for about 30 minutes. The resulting carboxyl groups on the PCL surface are reacted with N-(3-Dimethylaminopropyl)-N′-ethylcarbodiimide hydrochloride, N-hydroxysuccinimide, and N-(2-Aminoethyl)maleimide trifluoroacetate salt in an about 1:10:25:5 molar ratio. The functionalized PCL fiber is then cryomilled by freezing in liquid nitrogen and milling at about 6 cycles for about 1-minute milling periods and about 2-minute cool-down cycles. The resulting cryomilled fibers are filtered through an about 100-μm filter and lyophilized before use.

In one embodiment, CSPGs are acquired commercially from biological chicken sources. Additional sources of CSPGs and other inhibitors of axonal growth can also be used in place of these CSPGs.

The overall gel is synthesized by physically mixing MeHA, PEG-SH, and PCL fiber fragments to form the NHC. In one embodiment, the NHC formulation contains about 7 mg/mL MeHA, about 7 mg/mL PEG-SH, and about 8 mg/mL of PCL fibers. In an alternative formulation, the NHC may be mixed with CSPGs at a concentration of about 200 μg/mL. However, alternative formulations include adjusting the concentrations of all the components listed above or blends, analogs, derivatives, modifications, simplifications, and mixtures thereof. Further, alternative formulations may incorporate variations of conjugated, bioactive nanofibers, as discussed above, within the hydrogel, NHC, or IPN for increased display of bioactive components. These incorporated nanofibers may be fragmented using techniques included but not limited to cryomilling, syringe fragmentation, mesh fragmentation, sonication, lysis, or similar mechanical and chemical methods.

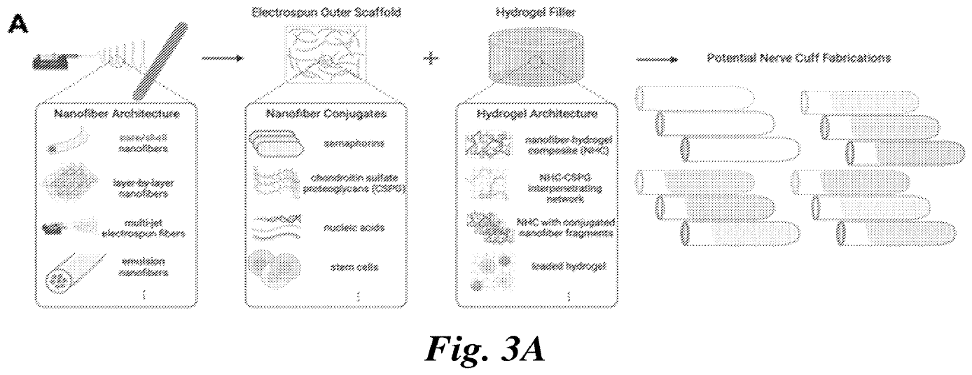

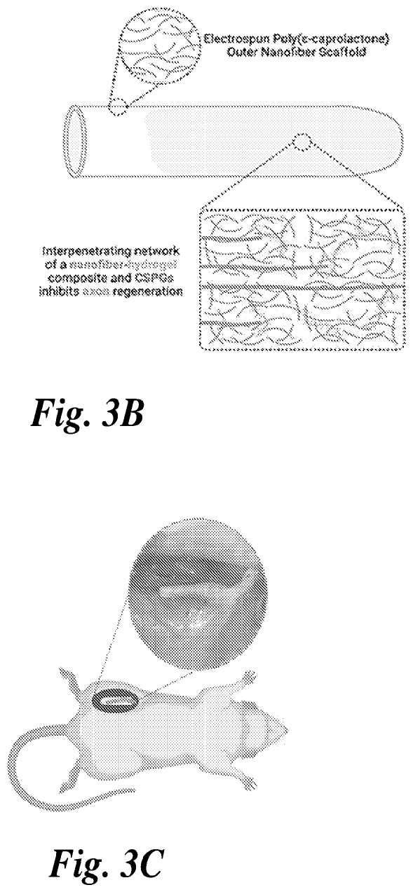

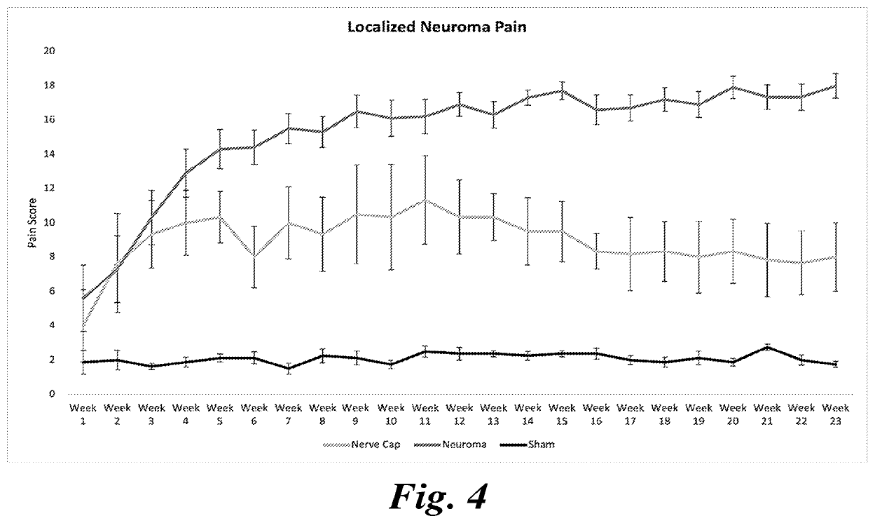

Thus, an example candidate library of nerve caps including functional components and a luminal filler is shown below with one embodiment of a cap in capping a severed rat sciatic nerve stump (FIG. 3).

### Example 3

### Behavioral Response to Mechanical Stimuli of the Neuroma

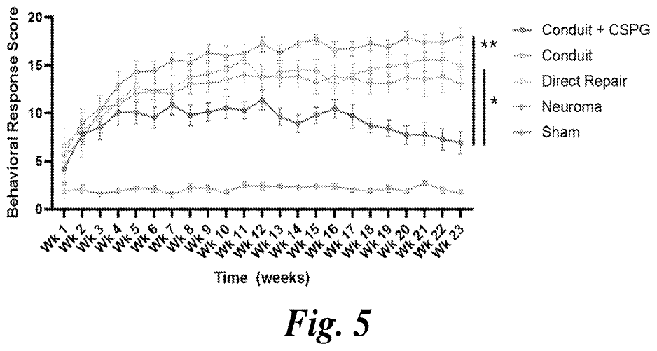

To evaluate the behavioral response to mechanical stimuli of the neuroma, a 15-G nylon monofilament was used to mechanically stimulate the area above the coaptation site. The nylon monofilament was applied to the area 10 times; each time for 2-3 seconds with a 1-2 minute interval in between trials. Responses were graded on a scale of 0-2 with a grade 0 being no response to the stimuli, a grade 1 response was defined as a slow withdrawal of the hind paw, and the grade 2 response consisted of a rapid withdrawal of the hind paw, licking of the area, shaking of the limb, or vocalization. The behavioral response score to evaluate neuroma pain was defined as the sum of responses for 10 trials, ranging from 0 to 20 with higher scores indicating greater pain. All behavioral tests were performed by a blinded examiner. See representative data provided in FIG. 5.

### Example 4

### Evaluation of the Regeneration of Axons Through the Conduit

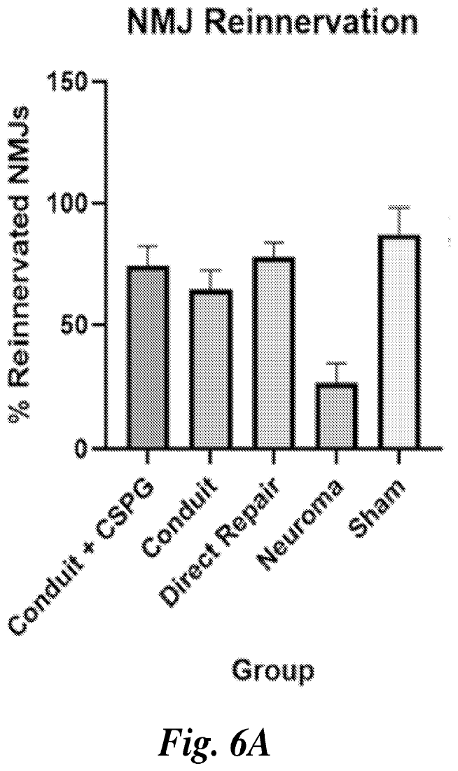

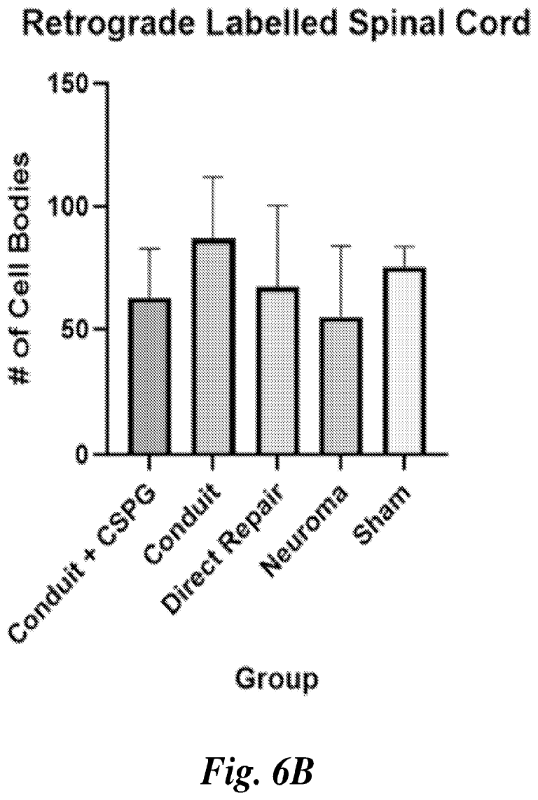

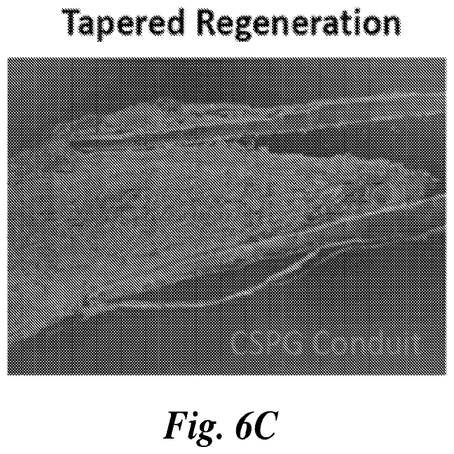

To evaluate the regeneration of axons through the conduit, target muscle reinnervation was assessed through immunofluorescence. The number of reinnervated neuromuscular junctions (NMJs) was calculated as the number of NMJs co-localized with neuronal marker beta-3-tubulin divided by the total number of NMJs in a section of lateral gastrocnemius muscle. No significant differences in reinnervation were seen across conduit and direct repair groups. The lateral gastrocnemius muscle was retrograde labelled with Di-I to assess the number of motor cell bodies contributing to target reinnervation. Similarly, no significant differences were seen across conduit and direct repair groups. To evaluate orientation of axonal growth through the conduit, longitudinal sections of the nerve in conduit were taken and stained with neurofilament. Tapered axonal growth and retention of CSPG within the conduit were observed in CSPG+Conduit animals. See representative data provided in FIG. 6

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Höke, A. Mechanisms of Disease: what factors limit the success of peripheral nerve regeneration in humans? Nat. Clin. Pract. Neurol. 2, 448-454 (2006).
Williams, H. B. The painful stump neuroma and its treatment. Clin. Plast. Surg. 11, 79-84 (1984).
Lewin-Kowalik, J., Marcol, W., Kotulska, K., Mandera, M. & Klimczak, A. Prevention and Management of Painful Neuroma. Neurol. Med. Chir. (Tokyo) 46, 62-68 (2006).
Cattell, R.B. & Ville, J. St. Amputation Neuromas of the Biliary Tract. Arch. Surg. 83, 242-246 (1961).
Scheib, J. & Höke, A. Advances in peripheral nerve regeneration. Nat. Rev. Neurol. 9, 668-676 (2013).
Chew, D. J., Fawcett, J. W. & Andrews, M. R. Chapter 14-The challenges of long-distance axon regeneration in the injured CNS. in Progress in Brain Research (eds. Dunnett, S. B. & Björklund, A.) vol. 201 253-294 (Elsevier, 2012).
Cregg, J. M. et al. Functional regeneration beyond the glial scar. Exp. Neurol. 253, 197-207 (2014).
Dellon, A. L., Mackinnon, S. E. & Pestronk, A. Implantation of Sensory Nerve into Muscle: Preliminary Clinical and Experimental Observations on Neuroma Formation. Ann. Plast. Surg. 12, 30-40 (1984).
Eberlin, K. R. & Ducic, I. Surgical Algorithm for Neuroma Management: A Changing Treatment Paradigm. Plast. Reconstr. Surg. Glob. Open 6, e1952 (2018).
Dumanian, G. A. et al. Targeted Muscle Reinnervation Treats Neuroma and Phantom Pain in Major Limb Amputees: A Randomized Clinical Trial. Ann. Surg. 270, 238-246 (2019).
Frey, M., Gruber, H., Holle, J. & Freilinger, G. An experimental comparison of the different kinds of muscle reinnervation: nerve suture, nerve implantation, and muscular neurotization. Plast. Reconstr. Surg. 69, 656-669 (1982).
Guth, L. & Zalewski, A. A. Disposition of cholinesterase following implantation of nerve into innervated and denervated muscle. Exp. Neurol. 7, 316-326 (1963).
Cheesborough, J. E., Smith, L. H., Kuiken, T. A. & Dumanian, G. A. Targeted Muscle Reinnervation and Advanced Prosthetic Arms. Semin. Plast. Surg. 29, 62-72 (2015).
Bushman, J. Localized immunosuppression of allografts for peripheral nerve repair. (2021).
Okuda, T. et al. The autotomy relief effect of a silicone tube covering the proximal nerve stump. J. Orthop. Res. 24, 1427-1437 (2006).
Dahlin, L. B. & Lundborg, G. Use of Tubes in Peripheral Nerve Repair. Neurosurg. Clin. N. Am. 12, 341-352 (2001).
Swanson, A. B., Boeve, N. R. & Lumsden, R. M. The prevention and treatment of amputation neuromata by silicone capping. J. Hand Surg. 2, 70-78 (1977).
Yan, H. et al. Mechanisms of Nerve Capping Technique in Prevention of Painful Neuroma Formation. PLOS ONE 9, e93973 (2014).
Nan, J., Hu, X., Li, H., Zhang, X. & Piao, R. Use of nerve conduits for peripheral nerve injury repair: A Web of Science-based literature analysis. Neural Regen. Res. 7, 2826-2833 (2012).
Höke, A. & Silver, J. Chapter 11 Proteoglycans and other repulsive molecules in glial boundaries during development and regeneration of the nervous system. in Progress in Brain Research (eds. Mize, R. R. & Erzurumlu, R. S.) vol. 108 149-163 (Elsevier, 1996).
Sandvig, A., Berry, M., Barrett, L. B., Butt, A. & Logan, A. Myelin-, reactive glia-, and scar-derived CNS axon growth inhibitors: Expression, receptor signaling, and correlation with axon regeneration. Glia 46, 225-251 (2004).
Derijck, A. A. H. A., Van Erp, S. & Pasterkamp, R. J. Semaphorin signaling: molecular switches at the midline. Trends Cell Biol. 20, 568-576 (2010).
Bashaw, G. J. & Klein, R. Signaling from Axon Guidance Receptors. Cold Spring Harb. Perspect. Biol. 2, a001941 (2010).
Sarhane, K. A. et al. Macroporous nanofiber wraps promote axonal regeneration and functional recovery in nerve repair by limiting fibrosis. Acta Biomater. 88, 332-345 (2019).
Ogle, R. C., III, E. A. B. & Neal, R. A. Compositions and methods for making and using laminin nanofibers. (2014).
Al-Enizi, A. M., Zagho, M. M. & Elzatahry, A. A. Polymer-Based Electrospun Nanofibers for Biomedical Applications. Nanomaterials 8, 259 (2018).
Painter, P. C. & Coleman, M. M. Essentials of polymer science and engineering. (DEStech Publications, Inc, 2008).
Bockelmann, J. et al. Functionalization of Electrospun Poly(ε-Caprolactone) Fibers with the Extracellular Matrix-Derived Peptide GRGDS Improves Guidance of Schwann Cell Migration and Axonal Growth. Tissue Eng. Part A 17, 475-486 (2011).
Li, X. et al. The effect of a nanofiber-hydrogel composite on neural tissue repair and regeneration in the contused spinal cord. Biomaterials 245, 119978 (2020).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A nerve cap for inhibiting neuroma formation and/or growth at a site of nerve injury, wherein the nerve cap has a tubular body having an open aperture proximate a severed end of the nerve and a cap distal the severed end of the nerve, wherein the tubular body has a shape defined by an outer wall, wherein an inner surface of the outer wall further defines a lumen, wherein the tubular body is configured to cover the severed end of the nerve at the site of injury.

2. The nerve cap of claim 1, wherein the outer wall comprises a nanofiber matrix comprising:
(a) one or more bioactive compounds;
(b) a plurality of nanofibers selected from electrospun nanofibers, including multi-jet electrospun nanofibers, core/shell nanofibers, layer-by-layer nanofibers, emulsion nanofibers, and combinations thereof;
(c) a semi-permeable matrix which allows for nutrient and fluid transport and immunomodulation via macrophage entrapment;
(d) has one or more characteristics selected from:
(i) a thickness ranging from about 50 μm to about 500 μm;
(ii) a pore size of less than about 10 μm;
(iii) a fiber diameter ranging from about 100 nm to about 2 μm; and
(iv) a plurality of randomly oriented nanofibers;
(e) one or more synthetic materials selected from poly(ε-caprolactone) (PCL), copolymers of ε-caprolactam and hexamethylendiaminadipate, polyglycolic acid (PGA), poly(lactic acid) (PLA), poly(1-lactic acid) (PLLA), copolymers of PLA and PGA, poly(lactic-co-glycolic acid) (PLGA), poly(vinyl acetate) (PVA), poly(ethylene-co-vinyl acetate) (PEVA), poly(ethylene glycol) (PEG), polyurethanes (PU), poly(ethylene oxide) (PEO), poly(vinyl pyrrolidone) (PVP), poly(ethylene terephthalate) (PET), poly(glycerol sebacate) (PGS), polydioxanone (PDO), polyphosphazenes (PPHOs), polyhydroxyalkanoates (PHA), polyhydroxybutyrates (PHB), polyhydroxyvalerate (PHV), polyhydroxyhexanoate (PHH), polyhydroxyoctanoate (PHO), and co-polymers, blends, analogs, derivatives, modifications, and mixtures thereof; and/or
one or more natural materials selected from hyaluronic acid (HA), silk, keratin, collagen, gelatin, fibrinogen, elastin, actin, myosin, cellulose, amylose, dextran, chitin, glycosaminoglycans (GAG), deoxyribonucleic acids (DNA), ribonucleic acids (RNA), chitin, chitosan (CS), alginate, and co-polymers, blends, analogs, derivatives, modifications, and mixtures thereof; and/or
(f) a plurality of core-shell structure nanofibers or a multilayered nanofiber assembly.

3-7. (canceled)

8. The nerve cap of claim 2, wherein the one or more bioactive molecules are encapsulated in the core-shell structure nanofibers or multilayered nanofiber assembly.

9. The nerve cap of claim 8, wherein the plurality of core-shell structures or multilayered nanofiber assembly are located at a predetermined location in the nanofiber matrix or are dispersed throughout the nanofiber matrix.

10. The nerve cap of claim 9, wherein the plurality of core-shell structures are located in the nanofiber matrix at the cap of the tubular body distal the severed end of the nerve.

11. The nerve cap of claim 1, wherein the lumen is open or the lumen comprises a hydrogel-based filler or a semi-interpenetrating network (IPN) filler.

12. (canceled)

13. The nerve cap of claim 11, wherein the hydrogel filler comprises an architecture selected from a nanofiber-hydrogel composite (NHC), an NHC interpenetrating network, an NHC comprising conjugated nanofiber fragments, and a loaded hydrogel.

14. The nerve cap of claim 13, wherein the NHC comprises functionalized poly(ε-caprolactone) (PCL) fiber fragments distributed in and covalently conjugated to a hydrogel network formed by reacting acrylated hyaluronic acid (HA) with thiolated poly(ethylene glycol) (PEG-SH).

15. The nerve cap of claim 11, wherein the hydrogel-based material or semi-interpenetrating network (IPN) filler inhibits axonal growth, polarizes macrophages to the pro-regenerative phenotype, supports angiogenesis, and combinations thereof.

16. The nerve cap of claim 11, wherein the hydrogel-based material:
(a) further comprises one or more bioactive agents covalently or non-covalently bond thereto;
(b) is selected from methacrylated hyaluronic acid (MeHA) crosslinked with thiolated poly(ethylene glycol) (PEG-SH), MeHA crosslinked with PEG-SH and chondroitin sulfate proteoglycans (CSPGs), MeHA cross-linked with PEG-SH, CSPGs, and PCL nanofiber fragments, a material that resembles native ECM, and combinations thereof, wherein the PEG-SH can be substituted by other chemical cross-linkers;
(c) is selected from a fibrin-, a collagen-, or a tissue matrix-derived hydrogel;
(d) comprises MeHA and CSPGs, and wherein the MeHA acts as a structural hydrogel that mimics native extracellular matrix (ECM), thereby resembling an environment of a peripheral nerve, and wherein the CSPGs inhibit axonal growth for preventing neuroma formation;
(e) comprises crosslinked PCL nanofiber fragments with hyaluronic acid;
(f) is physically mixed with CSPG to create a semi-interpenetrating network; and/or
(g) has an overall stiffness (shear storage modulus, G′) ranging from about 50 Pa to about 500 Pa.

17-22. (canceled)

23. The nerve cap of claim 2, wherein the one or more bioactive molecules comprising the nanofiber matrix and/or the hydrogel-based filler or a semi-interpenetrating network filler:
(a) recapitulate membrane bound signaling molecules;
(b) comprise one or more extracellular matrix components;
(c) inhibit axonal outgrowth;
(d) that inhibit axonal outgrowth are selected from a semaphorin, a myelin-associated glycoprotein, and one or more chondroitin sulfate proteoglycans (CSPGs); and/or
(e) are selected from a small molecule, a chondroitin sulfate proteoglycan (CSPG), a bioactive protein, an intracellular signaling pathway involved in growth cone collapse, such as a cyclic nucleotide, a nucleic acid, a bioactive molecule-secreting cell, a stem cell, an hiPSC-derived Schwann Cell, and combinations thereof.

24-27. (canceled)

28. The nerve cap of claim 23, wherein the semaphorin provides axon guidance during development by inhibiting growth cones.

29. The nerve cap of claim 2, wherein the one or more bioactive compounds are covalently bound to nanofibers of the nanofiber matrix.

30. The nerve cap of claim 29, wherein the nanofibers of the nanofiber matrix comprise PCL and the one or more bioactive compounds are conjugated onto the PCL nanofibers via grafted poly-(acrylic acid) (PAA).

31. The nerve cap of claim 1, wherein the outer wall of the tubular body has one or more surfaces having a morphology selected from a smooth morphology, a crimped morphology, and combinations thereof.

32. The nerve cap of claim 31, wherein the crimped morphology has a kink-resistance of up to a 90° bend and a length adjustability of less than or equal to 100% of an initial cap length.

33. The nerve cap of claim 1, wherein the nerve cap further comprises one or more suturable extensions, wherein the one or more suturable extensions have one or more dimensions ranging in diameter from about 50 μm to about 25 mm and in length from about 0 mm to about 50 mm.

34. (canceled)

35. The nerve cap of claim 1, wherein the nerve cap has one or more characteristics selected from:
(a) an open proximate end having a diameter ranging from about 0 mm to about 25 mm;
(b) a closed distal end having a shape selected from rounded, pointed, or cubic;
(c) a uniform diameter along a length of the cap or a gradient diameter along a length of the cap; and
(d) a length of the cap ranging from about 5 mm to about 50 mm.

36. A method for preparing a nerve cap having a predetermined shape, the method comprising:
(a) preparing or providing an outer nanofiber scaffold;
(b) deposing a portion of the outer nanofiber scaffold over a mold having a predetermined shape and having a tip of a predetermined shape;
(c) heating the outer nanofiber scaffold at a temperature sufficient to at least partially melt fibers of the outer nanofiber scaffold while the scaffold is deposed on the mold; and
(d) allowing the heated outer nanofiber scaffold to solidify to form the nerve cap.

37. The method of claim 36, wherein:
(a) the mold comprises a three-dimensional cylindrical mold; and/or
(b) the tip of the mold has a predetermined shape selected from a rounded tip, a pointed tip, and a cubic tip.

38. (canceled)

39. The method of claim 36, wherein the outer nanofiber scaffold is prepared by one or more methods selected from electrospinning a polymer solution, rotary jet spinning of a polymer solution, phase separation of a polymer solution, polymer nanofiber self-assembly, magnetospinning a polymer solution, and melt blowing a polymer melt.

40. The method of claim 39, wherein the electrospinning is conducted under one or more conditions selected from:
(a) a concentration of the polymer solution ranging from about 5 wt % to about 20 wt % of polymer;
(b) a molecular weight of the polymer ranging from 15,000 to about 100,000;
(c) an electrospinning voltage having a range from about 5 kV to about 24 kV;
(d) a distance from a polymer solution nozzle, needle, or spinneret to a rotating mandrel having a range from about 3 cm to about 20 cm; and
(e) a mandrel rotation speed having a range from about 50 RPM to about 1000 RPM.

41. The method of claim 40, wherein the polymer comprises one or more synthetic materials selected from poly(ε-caprolactone) (PCL), copolymers of ε-caprolactam and hexamethylendiaminadipate, polyglycolic acid (PGA), poly(lactic acid) (PLA), poly(1-lactic acid) (PLLA), copolymers of PLA and PGA, poly(lactic-co-glycolic acid) (PLGA), poly(vinyl acetate) (PVA), poly(ethylene-co-vinyl acetate) (PEVA), poly(ethylene glycol) (PEG), polyurethanes (PU), poly(ethylene oxide) (PEO), poly(vinyl pyrrolidone) (PVP), poly(ethylene terephthalate) (PET), poly(glycerol sebacate) (PGS), polydioxanone (PDO), polyphosphazenes (PPHOs), polyhydroxyalkanoates (PHA), polyhydroxybutyrates (PHB), polyhydroxyvalerate (PHV), polyhydroxyhexanoate (PHH), polyhydroxyoctanoate (PHO), and co-polymers, blends, analogs, derivatives, modifications, and mixtures thereof.

42. The method of claim 40, wherein the polymer is dissolved in an organic solvent selected from ethyl ether, hexane, tetrachloroethane, toluene, xylene, hexafluoro-2-propanol, and analogs, derivatives, modifications, and mixtures thereof.

43. The method of claim 39, wherein the method comprises electrospinning an about 8-wt % PCL solution using 80,000 molecular weight (MW) PCL in a 9:1 solvent of dichloromethane (DCM) and dimethylformamide (DMF) at about 10 kV at a 10-cm distance from a mandrel rotating at about 140 RPM.

44. The method of claim 36, further comprising functionalizing nanofibers of the outer nanofiber scaffold with one or more bioactive molecules; wherein:
(a) the one or more bioactive molecules are conjugated to the nanofiber;
(b) the one or more bioactive molecules are conjugated to the nanofiber through grafting;
(c) the grafting includes direct copolymerization, end functionalization, bulk grafting, solution grafting, use of an emulsion or mini-emulsion technique, use of supercritical conditions, gamma irradiation, electron beam, visible light exposure, ultraviolet irradiation, and mechanical shear; and/or
(d) the conjugation is introduced by the use of one or more of an enzyme, an alkene, an alkoxyamine, a ketone, a thiol, a norbornene, a propargyl, an alkyne, an azide, a carboxylic acid, and combinations, analogues, or derivatives thereof.

45-48. (canceled)

49. The method of claim 36, further comprising removing excess material beyond a sealed end of the nerve cap.

50. The method of claim 36, further comprising forming extensions or alterations to a surface morphology of the nerve cap by one or more of:
(a) electrospinning directly onto a mold with the desired structure;
(b) fabricating electrospun sheets to cover a mold;
(c) introducing a crimped surface morphology and associated techniques therein;
(d) using a negative mold or negative and positive mold combination for shaping; and
(e) changing the size or shape of the nerve cap mold.

51. The method of claim 36, further comprising at least partially filling a lumen of the nerve cap with a hydrogel-based filler material or a semi-interpenetrating network (IPN) filler, wherein the hydrogel-based filler material or a semi-interpenetrating network (IPN) filler can further comprise one or more bioactive compounds.

52. A method for inhibiting axon regeneration, promoting growth cone collapse, and/or preventing or inhibiting neuroma formation associated with a nerve injury, the method comprising capping an injured nerve of the subject with a nerve cap of claim 1; wherein:
(a) the closed distal end of the nerve cap provides a physical barrier to prevent or inhibit continued nerve growth;
(b) the nerve injury comprises a peripheral nerve injury;
(c) the nerve injury arises as a result of an amputation or another case of significant nerve injury; and/or
(d) the subject is an amputee.

53-56. (canceled)
